(19) United States
(12) Patent Application Publication
(10) Pub. No.: US 2014/0277196 A1
(43) Pub. Date: Sep. 18, 2014

(54) SPINAL IMPLANT SYSTEM AND METHOD

(71) Applicant: WARSAW ORTHOPEDIC, INC., Warsaw, IN (US)

(72) Inventors: Kevin T Foley, Germantown, TN (US); Gregory C. Marik, Collirville, TN (US); Trevor Seck, Memphis, TN (US); Jeff R. Justis, Germantown, TN (US)

(73) Assignee: WARSAW ORTHOPEDIC, INC., Warsaw, IN (US)

(21) Appl. No.: 13/804,484

(22) Filed: Mar. 14, 2013

(51) Int. Cl. A61B 17/70

(52) U.S. Cl. CPC A61B 17/7089
USPC 606/86 A

(57) ABSTRACT

A surgical guide comprises a frame connected with a first instrument that defines a first axis and extends to a tissue engaging surface. A guide element is connected with the frame and includes an inner surface that defines a passageway. The frame is rotatable relative to the first axis to translate the guide element along a trajectory that intersects the first axis such that an end of the guide element is angularly oriented relative to an orientation of the tissue engaging surface. At least one second instrument and/or at least one implant are movable along the passageway and relative to the inner surface to adjacent the tissue engaging surface. Systems and methods of use are disclosed.

### TECHNICAL FIELD

The present disclosure generally relates to medical devices for the treatment of musculoskeletal disorders, and more particularly to a surgical system for implant delivery to a surgical site and a method for treating a spine.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. As part of these surgical treatments, spinal constructs such as bone fasteners and vertebral rods are often used to provide stability to a treated region. For example, during surgical treatment, surgical instruments can be used to prepare a surgical site and bone fasteners can be delivered to the surgical site for fixation with bone to immobilize a joint. This disclosure describes an improvement over these prior art technologies.

### SUMMARY

In one embodiment, in accordance with the principles of the present disclosure, a surgical guide is provided. The surgical guide comprises a frame connected with a first instrument that defines a first axis and extends to a tissue engaging surface. A guide element is connected with the frame and includes an inner surface that defines a passageway. The frame is rotatable relative to the first axis to translate the guide element along a trajectory that intersects the first axis such that an end of the guide element is angularly oriented relative to an orientation of the tissue engaging surface. At least one second instrument and/or at least one implant are movable along the passageway and relative to the inner surface to adjacent the tissue engaging surface.

### BRIEF DESCRIPTION OF THE DRAWINGS

The present disclosure will become more readily apparent from the specific description accompanied by the following drawings, in which:

FIG. 1 is a perspective view of components of one particular embodiment of a system in accordance with the principles of the present disclosure;

FIG. 2 is a perspective view of the components of the system shown in FIG. 1;

FIG. 3 is a break away perspective view of components of the system shown in FIG. 1;

FIG. 4 is a break away perspective view of components of the system shown in FIG. 1;

FIG. 5 is a break away perspective view of components of the system shown in FIG. 1;

FIG. 6 is a break away perspective view of components of the system shown in FIG. 1;

FIG. 7 is a perspective view of components of one embodiment of a system in accordance with the principles of the present disclosure;

FIG. 8 is a perspective view of components of one embodiment of a system in accordance with the principles of the present disclosure;

FIG. 9 is a break away perspective view of components of the system shown in FIG. 8;

FIG. 10 is a perspective view of a component of the system shown in FIG. 8;

FIG. 11 is a perspective view of a component of the system shown in FIG. 8;

FIG. 12 is a side view of components of the system shown in FIG. 8 disposed with vertebrae;

FIG. 13 is a side view of components of the system shown in FIG. 8 disposed with vertebrae;

FIG. 14 is a side view of components of the system shown in FIG. 8 disposed with vertebrae;

FIG. 15 is a side view of components of the system shown in FIG. 8 disposed with vertebrae;

FIG. 16 is side view of components of system shown in FIG. 8 disposed with vertebrae;

FIG. 17 illustrates break away plan views of components of system shown in FIG. 8 disposed with vertebrae;

FIG. 18 is a perspective view of components of one embodiment of a system in accordance with the principles of the present disclosure;

FIG. 19 is a break away perspective view of components of the system shown in FIG. 18;

FIG. 20 is a perspective view of components of one embodiment of a system in accordance with the principles of the present disclosure;

FIG. 21 is perspective view of one embodiment of a system in accordance with the principles of the present disclosure disposed with vertebrae; and

FIG. 22 is a break away plan view of the system and vertebrae shown in FIG. 21.

### DETAILED DESCRIPTION

The exemplary embodiments of the surgical system and related methods of use disclosed are discussed in terms of medical devices for the treatment of musculoskeletal disorders and more particularly, in terms of a surgical system for implant delivery to a surgical site and a method for treating a spine. In one embodiment, the systems and methods of the present disclosure are employed with a spinal facet joint and fusion, for example, with a cervical, thoracic, and/or lumbar region of a spine. In one embodiment, the systems and methods can be employed with a spinal fusion, such as, for example, a posterior facet joint fusion.

In one embodiment, the system includes a guide tube with a curved drill guide for spinal fixation, such as, for example, cervical facet fixation. In one embodiment, the system includes a facet elevator and a percutaneous screw guide. In one embodiment, the elevator is disposable with the facet and the guide can introduce bone graft with a facet joint. In one embodiment, the elevator is disposable within the facet and the guide can introduce a screw across a facet joint. In one embodiment, the system includes an elevator/fork attachment that is cannulated to follow a guide wire into the facet joint after dilation of the facet joint.

In one embodiment, the system includes a guide having a scope for a posterior cervical procedure. The guide can perform a percutaneous screw placement utilizing a retractor tube with a pivoting drill, tap and screw guide. The system can include flexible instrumentation. The instrumentation can include a flexible trocar, a flexible drill bit, a flexible tap, a flexible self holding driver and/or a universal quick connect handle. In one embodiment, the system includes straight instrumentation, such as, for example, a retractor tube, a flexible arm, dilators and/or micro pituitary instruments.

In one embodiment, the system includes a guide and a facet elevator and/or a facet rasp. For example, the facet elevator can be used to elevate a facet joint during screw placement. For example, the facet rasp can be used for decortication of a facet joint. In one embodiment, the system includes at least one implant. In some embodiments, the implant may include bone screws, cortical screws, self tapping screws and/or self tapping hex driven screws. In one embodiment, the implant may include Herbert screws and/or reverse Herbert screws and/or double threaded screws for joint distraction and/or maintaining a joint space in an open orientation. In some embodiments, the screws may include screws of alternate size, for example, in a range of approximately 3.5 through 24 millimeters.

In one embodiment, the system includes a curved guide tube and a retractor tube. The retractor tube is attached to the curved guide tube by two pivoting points near the proximal end of the retractor tube. This configuration facilitates placing cervical facet screws along a trajectory of a cervical facet joint while avoiding difficult physical features and occipital bone. In some embodiments, this configuration provides access to joints higher in the spine, such as the cervical region, to reduce screw trajectory issues and allow the retractor tube to be positioned adjacent or across the facet joint space. In some embodiments, the facet joint space and the curved guide tube is disposed uniformly and consistently along a selected fixed trajectory of the cervical facet joint for orienting instruments and/or implants with the cervical facet joint for treatment. This configuration avoids realignment of the components of the system with the cervical facet joint, for example, having to repeat determination of screw trajectory with the cervical facet joint. In one embodiment, the curved guide tube rotates relative to the retractor tube and/or is detachable from the retractor tube. In one embodiment, the curved guide tube designates a surgical site, for example, by marking an incision area, and facilitates treatment of a facet joint utilizing flexible instrumentation. In one embodiment, the system is used to treat various sections of a spine and/or bone and is not limited to the treatment of a cervical facet joint.

In one embodiment, the system includes a curved pivoting guide that is capable of being attached and detached from instrumentation of the system. In one embodiment, the guide includes a straight section at its distal end to maintain linear motion of instruments. In one embodiment, the system includes components having a non-reflective coating. In one embodiment, the guide includes a locking mechanism. In one embodiment, the system includes a snake arm attached to the retractor tube. In one embodiment, the instrumentation may include spring loaded capturing devices. In one embodiment, the instrumentation may include a tapered hex tip for gripping a screw. In one embodiment, the instrumentation includes a drill bit that is an individual flexible instrument. The drill bit can include active stop or drill depth markings and may include a spring loaded distal end to guide drill passage in a center of the curved guide. In one embodiment, the system includes a facet elevator having a U shaped distraction guide. For example, the facet elevator maintains a facet in a selected position while drilling and inserting a screw. In one embodiment, the system includes a graft inserter device that facilitates inserting graft or an implant along the passageway into the facet joint. In one embodiment, the facet elevator is employed independently from a guide and is inserted into a surgical site via a cannula, dilator and/or retractor and/or an incision in a patient's body.

In one embodiment, the guide is attached to the instrumentation via a wing bolt that locks the guide to the tube. In one embodiment, the system includes a retractor tube having a fork style opening at its distal end. In one embodiment, the retractor tube includes a mating surface, such as, for example, ears for mating with the curved guide.

In one embodiment, the system includes a flexible capturing driver. In some embodiments, the driver may include a hex driver with capturing pivoting clasps, a flexible shaft and/or a quick connect proximal end. For example, the pivoting clasps allow the driver to hold onto screws while they are being passed along the passageway of the curved guide. The driver may be employed with tips attached thereto. In one embodiment, the driver includes a trocar tip that can be initially inserted with the guide through the incision and docked onto an inferior articular process. In one embodiment, the driver includes an awl tip that penetrates the articular process and creates an opening across the facet joint.

In one embodiment, the system includes a ratchet handle for selectively using the instrumentation. In one embodiment, the system includes a straight facet rasp.

In one embodiment, the systems may be employed with a method that comprises the steps of determining facet joint trajectory; dilating the facet joint; placing a retractor tube in the facet joint; removing soft tissue and decorticating vertebral surfaces; inserting a percutaneous guide in the facet joint; and drilling across the facet joint.

In one embodiment, the systems may be employed with a method that comprises the steps of determining facet joint trajectory visually for example in an open surgical procedure, or through the use of a fluoroscope or image guidance such as, for example, an O-arm and/or surgical navigation. In one embodiment, the step of distracting includes spacing the facet joint with a ramp disposed at an end of the guide. In one embodiment, the method further comprises the step of depositing graft and/or an implant at the facet joint. In one embodiment, the step of depositing includes inserting a PEEK spacer with the facet elevator.

In one embodiment, the systems may be employed with a method for treating bone such as, for example, a spine that includes providing a surgical retractor tube and a pivoting curved drill guide. The method is employed to percutaneously deliver a screw along a passageway of the guide after docking the components of the system and decorticating a joint through the surgical retractor tube. The method includes the steps of providing flexible instrumentation to work along the passageway of the curved guide and screw(s) along the passageway.

In one embodiment, the systems may be employed with a method that comprises the steps of determining a midline on a body adjacent a surgical site, for example, adjacent C6-C7 level of cervical vertebrae as a target trajectory; and determining a trajectory, for example, using a 20-gauge spinal needle to determine the trajectory using physical landmarks and fluoroscopy and/or image guidance, such as, for example, surgical navigation simultaneously. In one embodiment, the trajectory is parallel and inline with the selected facet joint leading up to the intersection of articular processes about 1-2 centimeters off midline, with the articular cartilage as the ending point. In one embodiment, the method further comprises making an incision for components of the system. In one embodiment, the method further comprises cutting the fascia to prepare for dilators. In one embodiment, the method further comprises inserting dilators to retract tissue for a tubular retractor; and sliding the retractor tube down to rest across the facet joint. A larger dimension dilator can be used to finally position the retractor tube and a flexible arm connected to the retractor tube can be tightened to fix position of the retractor tube. In one embodiment, the systems described can be employed with a fully open surgical procedure.

In one embodiment, the systems described can be employed in a hybrid surgical procedure where an incision is used for inserting an elevator and the guide tube delivering the screw. In one embodiment, the system includes a guide having a facet elevator including tangs disposed at an angle relative to an axis of the elevator, for example, 45 degrees. In some embodiments, the tangs may be aligned via alternate orientations, such as, for example, offset, staggered, irregular, uniform, non-uniform and/or relative angular orientations including acute, perpendicular and obtuse. In one embodiment, the tangs may be configured as a wedge.

In one embodiment, the method further comprises removing and decorticating soft tissue and/or articular cartilage from the vertebral surfaces using rongeurs, reamers and/or rasps in preparation for implantation of a facet screw; attaching the curved guide to the retractor tube and indicating location of the incision point; making an incision to allow the curved guide to enter through the skin of the body; inserting the curved guide along a selected screw trajectory of a cervical facet joint; inserting a flexible capturing driver and interchangeable trocar tip into the curved guide; driving the curved guide through the incision and soft tissue while the trocar tip allows it to pass through the tissue; resting the distal end of the guide on the inferior articular process; and locking the curved guide in place. In one embodiment, the method further comprises removing the flexible driver and trocar from the curved guide and the surgical site; attaching an awl tip to the flexible driver, inserting the driver with the awl tip in the curved guide and passing the driver along the passageway to the facet joint; and drilling across the facet joint to drill a screw hole.

In one embodiment, the systems may be employed with a method that comprises the steps of placing a retractor tube over a dilator over a facet. A practitioner can work down the retractor tube to open the facet capsule and decorticate the facet and place graft material. The method includes the step of inserting a curved dilator inside the curved guide. The curved assembly is inserted through an extension of the retractor tube in a radial fashion coming to rest at the end of the retractor tube, perpendicular to the facet and the retractor tube. The method includes the step of removing the curved dilator from the curved guide; and inserting a flexible drill shaft with a drill tip through the curved guide to drill across the facet. The practitioner may monitor the surgical site visually through the retractor tube, for example, to view drilling and joining of the facet joint. The method includes the step of translating a screw along the passageway of the curved guide with a flexible driver across the facet; removing the curved guide; and removing the retractor tube.

In one embodiment, one or all of the components of the surgical system are disposable, peel-pack, pre-packed sterile devices used with an implant. One or all of the components of the surgical system may be reusable. The surgical system may be configured as a kit with multiple sized and configured components.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In one embodiment, the present disclosure may be employed with other osteal and bone related applications, including those associated with diagnostics and therapeutics. In some embodiments, the disclosed surgical system and methods may be alternatively employed in a surgical treatment with a patient in a prone, supine position, lateral and/or employ various surgical approaches to the spine, including anterior, posterior, posterior mid-line, lateral, postero-lateral, and/or antero-lateral approaches, and in other body regions. The present disclosure may also be alternatively employed with procedures for treating the lumbar, cervical, thoracic, sacral and pelvic regions of a spinal column. The system and methods of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

The following discussion includes a description of a surgical system and related methods of employing the surgical system in accordance with the principles of the present disclosure. Alternate embodiments are also disclosed. Reference will now be made in detail to the exemplary embodiments of the present disclosure, which are illustrated in the accompanying figures. Turning now to FIGS. 1-6, there is illustrated components of a surgical system, such as, for example, a spinal implant system 20 in accordance with the principles of the present disclosure.

The components of spinal implant system 20 can be fabricated from biologically acceptable materials suitable for medical applications, including metals, synthetic polymers, ceramics and bone material and/or their composites, depending on the particular application and/or preference of a medical practitioner. For example, the components of spinal implant system 20, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate such as hydroxyapatite (HA), corraline HA, biphasic calcium phosphate, tricalcium phosphate, or fluorapatite, tri-calcium phosphate (TCP), HA-TCP, calcium sulfate, or other resorbable polymers such as polyaetide, polyglycolide, polytyrosine carbonate, polycaroplaetohe and their combinations, biocompatible ceramics, mineralized collagen, bioactive glasses, porous metals, bone particles, bone fibers, morselized bone chips, bone morphogenetic proteins (BMP), such as BMP-2, BMP-4, BMP-7, rhBMP-2, or rhBMP-7, demineralized bone matrix (DBM), transforming growth factors (TGF, e.g., TGF-β), osteoblast cells, growth and differentiation factor (GDF), insulin-like growth factor 1, platelet-derived growth factor, fibroblast growth factor, or any combination thereof.

Various components of spinal implant system 20 may have material composites, including the above materials, to achieve various desired characteristics such as strength, rigidity, elasticity, compliance, biomechanical performance, durability and radiolucency or imaging preference. The components of spinal implant system 20, individually or collectively, may also be fabricated from a heterogeneous material such as a combination of two or more of the above-described materials. The components of spinal implant system 20 may be monolithically formed, integrally connected or include fastening elements and/or instruments, as described herein.

Spinal implant system 20 is employed, for example, with a minimally invasive procedure, including percutaneous techniques, mini-open and open surgical techniques to deliver and introduce instrumentation and/or an implant, such as, for example, a bone fastener, at a surgical site within a body of a patient, for example, a section of a spine. In one embodiment, spinal implant system 20 and related methods may be employed with treatments using minimally invasive and percutaneous techniques. In one embodiment, spinal implant system 20 includes an implant having a horseshoe configuration for disposal about a bone screw and/or other bone fasteners such that the horseshoe shape is disposed within the facet joint space. In one embodiment, the implant is initially disposed with a facet joint space and includes an opening configured for disposal of a bone screw and/or a bone fastener.

Spinal implant system 20 includes a surgical guide 22 configured to facilitate disposal of working instruments and/or a spinal implant adjacent to a surgical site, as described herein. Guide 22 includes a frame 24 extending between a first end 26 and a second end 28. End 26 includes an inner surface that defines an opening 27 and is connected with a guide element, as described herein. Opening 27 is threaded for releasable fixation with surgical instruments, as described herein. In one embodiment, one or all of the surfaces of frame 24 may have alternate surface configurations, such as, for example, smooth, rough, threaded for connection with surgical instruments, arcuate, undulating, porous, semi-porous, dimpled, polished and/or textured according to the requirements of a particular application.

Frame 24 includes spaced apart bifurcated arms, such as, for example, a first arm 30 and a second arm 32. Arm 30 extends between a proximal end 34 and a distal end 36. An intermediate portion 38 is disposed between ends 34, 36. End 34 is connected with and monolithically formed with end 26. In one embodiment, end 34 may be integrally connected and/or fastened with end 26.

Portion 38 includes a lateral opening 40. Opening 40 is configured for disposal of a tensioning member, as described herein. In one embodiment, opening 40 may have various configurations, such as, for example, circular, oval, rectangular, polygonal, irregular, tapered, offset, staggered, uniform and non-uniform.

Arm 32 extends between a proximal end 42 and a distal end 44. An intermediate portion 46 extends between ends 42, 44. End 42 is connected with and monolithically formed with end 26. In one embodiment, end 42 may be integrally connected and/or fastened with end 26. In some embodiments, arms 30, 32 are disposed in a substantially parallel orientation. In one embodiment, arms 30, 32 may be disposed in alternate orientations, such as, for example, converging, diverging, offset, staggered and/or relative angular orientations including acute, perpendicular and obtuse.

Arm 32 includes a hinge 48 disposed adjacent to end 42. Hinge 48 includes a pin 49 that connects arm 32 with end 42 and is configured to pivot arm 32 relative to frame 24 for rotation relative thereto. Hinge 48 pivots arm 32 into engagement with a surgical instrument such that frame 24 captures the surgical instrument, as will be described.

Portion 46 includes a lateral opening 50. Opening 50 is configured for disposal of the tensioning member. The tensioning member includes an end fixed within opening 50 such that arm 30 can be drawn toward arm 32 for capturing a surgical instrument, as will be described.

Frame 24 includes a tensioning latch that includes a tensioning member 52 disposed transverse to the relative orientation of arms 30, 32. Member 52 connects arms 30, 32 in a relatively movable orientation. Member 52 extends between a first portion 54 and a second portion 56.

Portion 54 is configured for disposal with and adjacent opening 40. The tensioning latch includes a latch 58 and a pin 60 that connects member 52 with latch 58. Latch 58 is configured to pivot about pin 60 and relative to member 52 for rotation relative thereto. Latch 58 is rotatable about pin 60 between a non-locking and a locking orientation through an angle of 180 degrees to tension member 52 such that arm 30 can be drawn toward arm 32 for capturing a surgical instrument and releasably locking frame 24 with the surgical instrument. Portion 56 is configured for disposal with and adjacent opening 50. Portion 56 is fixed with arm 32 via a pin 62 to facilitate tensioning of arms 30, 32. In one embodiment, the tensioning latch is biased to the non-locking orientation and/or the locking orientation by, for example, a spring connected with latch 58.

Frame 24 includes a mating surface, such as, for example, a connector 64. Connector 64 is configured to capture and release a surgical instrument. Connector 64 includes a female mating part 65, which includes an ear 66 and an ear 68. Ear 66 is disposed at end 36 of arm 30 and ear 68 is disposed at end 44 of arm 32. Ears 66, 68 each have a cup configuration and are inwardly oriented for a capture engagement with a surgical instrument, as described herein. Ears 66, 68 include inner surfaces 70, 72 respectively, which define cavities for disposal of male mating parts, such as, for example, a cap, disc and/or knob of a surgical instrument that is releasably disposable within the cavities of ears 66, 68. It is envisioned that frame 24 may include one or a plurality of mating surfaces.

In one embodiment, connector 64 can include a male mating part and the surgical instrument can include a female mating part. In some embodiments, mating parts can comprise a pin, bracket, hook, clip and/or key/keyway engagement. A lever 74 is connected to ear 68. Lever 74 includes a capturing element, such as, for example, a socket drive, pin, bracket, hook, clip and/or key/keyway for releasable fixation with a surgical instrument. Lever 74 is rotatable about ear 68 to actuate the capturing element of lever 74 for releasable capture of a surgical instrument.

Guide 22 includes a guide element, such as, for example, a sleeve 76. Sleeve 76 is connected with and monolithically formed with end 26. Sleeve 76 has an arcuate configuration and extends between a first end 78 and a second end 80 that defines a distal face including an opening configured for working passage of a surgical instrument and/or an implant. In one embodiment, sleeve 76 is integrally connected and/or fastened with end 26. In some embodiments, sleeve 76 may include alternate configurations, such as, for example, linear, offset, staggered and/or an angular orientation.

Sleeve 76 includes an inner surface 82 that defines a passageway 84 having an arcuate configuration. Passageway 84 is configured such that a surgical instrument and/or at least one implant, such as, for example, a bone screw 85 is disposable and/or translatable along passageway 84 relative to inner surface 82 along a selected trajectory. A surgical instrument and/or an implant is introduced into opening 27 and passageway 84 is configured as a working channel such that the surgical instrument and/or implant are translated therethrough and delivered through end 80 to adjacent a surgical site for treating vertebrae. In one embodiment, passageway 84 may have alternate configurations, such as, for example, oval, oblong, triangular, square, polygonal, irregular, uniform, non-uniform, offset, staggered, undulating, variable, V-shaped, and/or tapered depending upon the geometry of the instrument and/or implant to be translated along passageway 84 and/or according to a selected trajectory.

System 20 includes a first instrument, such as, for example, a facet elevator 86 connected with frame 24. Elevator 86 extends between a first portion 88 and a second portion 90, and defines a first axis a. Portion 88 includes a gripping portion 92. Gripping portion 92 includes a gripping surface for manipulation of gripping portion 92 and may be variously configured according to the requirements of a particular application. In one embodiment, elevator 86 is employed for treating vertebrae during a surgical procedure, for example, to lift, distract and/or separate tissue, such as, for example, soft tissue, bone, vessels and organs, adjacent a surgical site.

Portion 90 defines a tissue engaging surface 94. Engaging surface 94 is configured to engage tissue and/or adjacent tissue, such as, for example, a cervical facet. Engaging surface 94 includes a first extension 96 and a second extension 98. In one embodiment, extensions 96, 98 are tapered. In some embodiments, extensions 96 and 98 are disposed at an angle relative to axis a of elevator 86 such as, for example, a 45° angle. In some embodiments, extensions 96, 98 may be aligned via alternate orientations, such as, for example, offset, staggered, irregular, uniform, non-uniform and/or relative angular orientations including acute, perpendicular and obtuse.

In one embodiment, as shown in FIG. 4, engaging surface 94 includes a wedge 97 configured for alignment with end 80 of sleeve 76. Wedge 97 has a ramp configuration for distraction and manipulation of a bone, such as, for example, the surfaces of a facet joint. In some embodiments, at least a portion of wedge 97 is monolithic and/or bifurcated. In one embodiment, wedge 97 has an inclined configuration at an angle α, such as, for example, a 45 degrees relative to a surface of wedge 97. In some embodiments, wedge 97 and elevator 86 are monolithically formed.

In one embodiment, as shown in FIG. 6, wedge 97 is releasably engageable with elevator 86 such that wedge 97 can be selectively removed from elevator 86. For example, wedge 97 can include a threaded shaft 99 that is configured for fixation within a threaded opening 101 positioned within portion 90. In some embodiments, portion 99 and opening 101 engage via a pin, bracket, hook, clip, male/female and/or key/keyway engagement.

Elevator 86 includes an inner surface that defines a passageway 102. Passageway 102 is configured for disposal of a guidewire (not shown) to facilitate translation of elevator 86 to adjacent a surgical site and/or tissue. In one embodiment, as shown in FIG. 5, passageway 102 is configured for disposal of a plunger 103 and a graft material 105. Plunger 103 is manipulable and configured for translation along an opening 107 to facilitate delivery of graft material 105 to the surgical site and/or the tissue. Opening 107 is located at a distal end of passageway 102 and is disposed between extensions 96 and 98. In some embodiments, opening 107 may be variously configured such as, for example, in a round, oval, oblong, triangular, square, polygonal, irregular, uniform, non-uniform, offset, staggered, undulating, variable, V-shaped, and/or tapered shape depending upon the geometry of the graft material selected.

Elevator 86 includes a mating surface, such as, for example, a connector 104. Connector 104 includes male mating parts, such as, for example, discs 106, 108 that are outwardly oriented and releasably engageable with ears 66, 68. Disc 106 is oriented for disposal within the cavity of ear 66 and disc 108 is oriented for disposal within the cavity of ear 68. The outer surface of disc 106 engages inner surface 70 and the outer surface of disc 108 engages inner surface 72 such that discs 106, 108 are freely rotatable relative to ears 66, 68. In one embodiment, the outer surfaces of discs 106, 108 engage surfaces 70, 72 for rotation via sliding surfaces, friction, key/keyway and/or bearing surfaces.

Lever 74 is rotated about ear 68 to actuate the capturing element of lever 74 for releasable capture of ear 68. Latch 58 is rotated about pin 60 and relative to member 52 for rotation relative thereto from a non-locking to a locking orientation to tension member 52 such that arm 30 can be drawn toward arm 32. As such, arms 30, 32 cause connectors 64, 104 to mate and releasably lock frame 24 with elevator 86. Ears 66, 68 are releasably fixed with discs 106, 108 and freely rotatable thereabout.

Frame 24 is rotated relative to axis a of elevator 86 such that arms 30, 32 pivot about connector 104. Sleeve 76 translates along an arcuate trajectory T that intersects axis a. End 80 translates along trajectory T in an orientation for disposal of end 80 at an angular orientation, such as, for example, substantially perpendicular, relative to an orientation of engaging surface 94. In some embodiments, end 80 may be disposed adjacent and/or in contacting engagement with surface 94 adjacent tissue and/or a surgical site.

With end 80 disposed substantially perpendicular to an orientation of engaging surface 94, screw 85 and/or a second surgical instrument, such as those examples described herein can be translated along passageway 84 and relative to inner surface 82 to adjacent end 80. The second surgical instrument is translated through sleeve 76 for treating vertebrae adjacent end 80. Screw 85 can be translated through sleeve 76 with an instrument, such as a driver, for fixation with vertebrae.

In one embodiment, as shown in FIGS. 7-17, spinal implant system 20, similar to the system and methods described above with regard to FIGS. 1-6, includes a surgical guide 122. Guide 122 includes a frame 124 extending between a first end 126 and a second end 128. End 126 includes an inner surface that defines an opening 127 and is connected with a guide element, as described herein. Opening 127 is threaded for releasable fixation with surgical instruments, as described herein.

Frame 124 includes spaced apart bifurcated arms, such as, for example a first arm 130 and a second arm 132. Arm 130 extends between a proximal end 134 and a distal end 136. An intermediate portion 138 is disposed between ends 134, 136. End 134 is connected with and monolithically formed with end 126. In one embodiment, end 134 may be integrally connected and/or fastened with end 126.

Portion 138 includes a lateral opening 140. Opening 140 is configured for disposal of a tensioning member, as described herein. Arm 132 extends between a proximal end 142 and a distal end 144. An intermediate portion 146 extends between ends 142, 144. End 142 is connected with and monolithically formed with end 126.

Arm 132 includes a hinge 148 disposed adjacent to end 142. Hinge 148 includes a pin 149 that connects arm 132 with end 142 and is configured to pivot arm 132 relative to frame 124 for rotation relative thereto. Hinge 148 pivots arm 132 into engagement with a surgical instrument such that frame 124 captures the surgical instrument, as will be described.

Portion 146 includes a lateral opening 150. Opening 150 is configured for disposal of the tensioning member. The tensioning member includes an end fixed within opening 150 such that arm 130 can be drawn into proximity with arm 132 for capturing a surgical instrument, as will be described.

Frame 124 includes a tensioning latch that includes a tensioning member 152 disposed transverse to the relative orientation of arms 130, 132. Member 152 connects arms 130, 132 in a relatively movable orientation. Member 152 extends between a first portion 154 and a second portion 156.

Portion 154 is configured for disposal with and adjacent opening 140. The tensioning latch includes a latch 158 and a pin 160 that connects member 152 with latch 158. Latch 158 is configured to pivot about pin 160 between a non-locking and a locking orientation through an angle of 180 degrees to tension member 152 such that arm 130 can be drawn toward arm 132 for capturing a surgical instrument and releasably locking frame 124 with the surgical instrument. Portion 156 is configured for disposal with and adjacent opening 150. Portion 156 is fixed with arm 132 via a pin 162 to facilitate tensioning of arms 130, 132. In one embodiment, the tensioning latch is biased to the non-locking orientation and/or the locking orientation by, for example, a spring connected with latch 158.

Frame 124 includes a mating surface, such as, for example, a connector 164. Connector 164 is configured to capture and release a surgical instrument. Connector 164 includes a female mating part 165, which includes an ear 166 and an ear 168. Ear 166 is disposed at end 136 of arm 130 and ear 168 is disposed at end 144 of arm 132. Ears 166, 168 each have a cup configuration and are inwardly oriented for a capture engagement with a surgical instrument, as described herein. Ears 166, 168 include inner surfaces 170, 172 respectively, which define cavities for disposal of male mating parts, such as, for example, a cap, disc and/or knob of a surgical instrument that is releasably disposable within the cavities of ears 166, 168.

A thumbwheel 174 is connected to ear 168. Thumbwheel 174 includes a capturing element, such as, for example, a socket drive, pin, bracket, hook, clip and/or key/keyway for releasable fixation with a surgical instrument. Thumbwheel 174 is rotatable about ear 168 to actuate the capturing element of thumbwheel 174 for releasable capture of a surgical instrument.

Guide 122 includes a guide element, such as, for example, a sleeve 176. Sleeve 176 is connected with and monolithically formed with end 126. Sleeve 176 has an arcuate configuration and extends between a first end 178 and a second end 180 that defines a distal face including an opening configured for working passage of a surgical instrument and/or an implant. In one embodiment, sleeve 176 is integrally connected and/or fastened with end 126.

Sleeve 176 includes an inner surface 182 that defines a passageway 184 having an arcuate configuration. Passageway 184 is configured such that a surgical instrument, such as, for example, a flexible driver 183, as shown in FIG. 11, and/or at least one implant, such as, for example, a bone screw 185 is disposable and/or translatable along passageway 184 relative to inner surface 182 along a selected trajectory. A surgical instrument and/or an implant is introduced into opening 127 and passageway 184 is configured as a working channel such that the surgical instrument and/or the implant are translated therethrough and delivered through end 180 to adjacent a surgical site for treating vertebrae. In some embodiments, passageway 184 may have alternate configurations, such as, for example, oval, oblong, triangular, square, polygonal, irregular, uniform, non-uniform, offset, staggered, undulating, variable, V-shaped, and/or tapered depending upon the geometry of the spinal construct to be received within passageway 184 and/or according to a selected trajectory.

System 20 includes a first instrument, such as, for example, a retractor tube 186, as shown in FIGS. 7-10, connected with frame 124. Tube 186 extends between a first portion 188 and a second portion 190, and defines a first axis b. Portion 188 includes an opening 192. Tube 186 via opening 192, for example, is configured to facilitate direct visualization of a surgical site and retraction of tissue adjacent to the surgical site. Portion 188 includes an extension, such as, for example, a snake arm 191 for connection to a fixed surface, such as, for example, surgical assemblies, table or other structure.

Portion 190 defines a tissue engaging surface 194. Engaging surface 194 includes an angled face and is configured to engage tissue and/or adjacent tissue, such as, for example, a cervical facet. Engaging surface 194 includes a wall surface that defines a cavity, such as, for example, concave opening 200. Opening 200 is configured for disposal of end 180 such that end 180 can be removably supported by tube 186. Tube 186 includes an inner surface that defines a passageway 202 that communicates with opening 192. It is envisioned that passageway 202 may be variously configured, similar to those alternatives described herein.

Tube 186 includes a mating surface adjacent portion 188, such as, for example, a connector 204. Connector 204 includes male mating parts, such as, for example, discs 206, 208 that are outwardly oriented and releasably engageable with ears 166, 168. Disc 206 is oriented for disposal within the cavity of ear 166 and disc 208 is oriented for disposal within the cavity of ear 168. The outer surface of disc 206 engages inner surface 170 and the outer surface of disc 208 engages inner surface 172 such that discs 206, 208 are freely rotatable relative to ears 166, 168. In some embodiments, the outer surfaces of discs 206, 208 engage surfaces 170, 172 for rotation via sliding surfaces, friction, key/keyway and/or bearing surfaces.

In operation, tube 186 is disposed such that ears 166, 168 are disposed in alignment with discs 206, 208. Thumbwheel 174 is rotated about ear 168 to actuate the capturing element of thumbwheel 174 for releasable capture of ear 168, as described above. Latch 158 is rotated about pin 160 and relative to member 152 for rotation relative thereto from a non-locking to a locking orientation to tension member 152 such that arm 130 can be drawn toward arm 132. As such, arms 130, 132 cause connectors 164, 204 to mate and releasably lock frame 124 with tube 186. Ears 166, 168 are releasably fixed with discs 206, 208 and freely rotatable thereabout. To manipulate and deliver a second surgical instrument and/or an implant to a surgical site, guide 122 is positioned adjacent the surgical site.

Frame 124 is rotated relative to axis b of tube 186 such that arms 130, 132 pivot about connector 204. Sleeve 176 translates along an arcuate trajectory T that intersects axis b. End 180 translates along trajectory T into an orientation for disposal of end 180 angular such as, for example, substantially perpendicular to an orientation of engaging surface 194. End 180 is disposed with cavity 200 and supported therein by tube 186. It is envisioned that support of end 180 by tube 186 provides a relatively fixed surface and stability such that passageway 184 provides a stabile working channel for surgical instrument and/or implants.

With end 180 disposed substantially perpendicular to an orientation of engaging surface 194, screw 185 and/or a second surgical instrument, such as flexible driver 183 can be translated along passageway 184 and relative to inner surface 182 to adjacent end 180. The second surgical instrument is translated through sleeve 176 for treating vertebrae adjacent end 180. Screw 185 can be translated through sleeve 176 with an instrument, such as a driver 183 for fixation with vertebrae.

In assembly, operation and use, as shown in FIGS. 12-17, spinal implant system 20, similar to that described herein, is employed with a surgical procedure for treatment of a spinal disorder affecting a section of a spine of a patient, as discussed herein. Spinal implant system 20 may also be employed with other surgical procedures. For example, spinal implant system 20 can be used with a surgical procedure for treatment of a condition or injury of an affected cervical section of the spine including vertebrae.

In use, to treat the affected section of vertebrae V, a medical practitioner obtains access to a surgical site including cervical vertebrae V1, V2, V3, as shown in FIG. 12, in any appropriate manner, such as through incision and retraction of tissues. It is envisioned that spinal implant system 20 may be used in any existing surgical method or technique including open surgery, mini-open surgery, minimally invasive surgery, and percutaneous surgical implantation, whereby vertebrae is accessed through a micro-incision, or sleeve that provides a protected passageway to the area. Once access to the surgical site is obtained, the particular surgical procedure is performed for treating the spinal disorder. Spinal implant system 20 is then employed to augment the surgical treatment. Spinal implant system 20 can be delivered or implanted as a pre-assembled device or can be assembled in situ. Spinal implant system 20 may be completely or partially revised, removed or replaced, for example, removing frame 124 and/or sleeve 176, tube 186 and/or one or all of the components of spinal implant system 20 before, during or after the surgical procedure.

A trajectory T (FIG. 16) is defined for insertion of screw 185 within a cervical section of a spine, as shown in FIG. 9. In one embodiment, trajectory T is determined using physical landmarks and fluoroscopy simultaneously. In one embodiment, trajectory T is parallel and inline with the appropriate joint leading up to the intersection of articular processes, with the articular cartilage being the ending point. It is envisioned that screw 185 may be inserted via a trajectory oriented from an anterior, posterior, superior and/or inferior direction. An incision is made and fascia is cut to prepare for a dilator/delivery tube. In one embodiment, a guide wire and/or a trocar-cannula assembly may be employed as an instrument for gaining access to the surgical site and/or defining the trajectory T.

A surgical passageway is created and a dilator/delivery tube (not shown) may be employed to deliver screw 185 to the surgical site, as shown in FIG. 12. It is envisioned that the dilator/delivery tube may be configured as an in-situ guidable instrument, and may include an endoscope camera tip for viewing insertion trajectory. Soft tissue is removed from facet joint FJ and decortication is performed via various instrumentation, such as, for example, a reamer (not shown) and/or a rasp 189, as shown in FIG. 13.

Spinal implant system 20 is disposed adjacent vertebrae V at a surgical site, as shown in FIGS. 15-17, and the components of spinal implant system 20 are manipulable to drive, torque, insert or otherwise connect screw 185 to vertebrae, according to the particular requirements of the surgical treatment.

Tube 186 is disposed in alignment with frame 124 and thumbwheel 174 is rotated about ear 168 to actuate the capturing element of thumbwheel 174 for releasable capture of ear 168, as described above. Latch 158 is rotated from a non-locking to a locking orientation to tension member 152 such that arm 130 can be drawn toward arm 132. Connectors 164, 204 mate and releasably lock frame 124 with tube 186.

Frame 124 is rotated relative to axis b of tube 186 such that arms 130, 132 pivot about connector 204. Sleeve 176 translates along trajectory T that intersects axis b. End 180 translates along trajectory T into an orientation for disposal of end 180 at an angular orientation, such as, for example, substantially perpendicular, relative to an orientation of engaging surface 194. End 180 is disposed with cavity 200 and supported therein by tube 186.

In one embodiment, with end 180 disposed substantially perpendicular to an orientation of engaging surface 194, a flexible capturing driver (not shown) and a trocar (not shown) are inserted into sleeve 176 and passed through the incision and soft tissue, as shown in FIG. 15. Sleeve 176 is advanced to the inferior articular process IAP and the driver and the trocar are removed. The driver comprising a drill tip 187 is inserted into sleeve 176 and pilot holes or the like are made in vertebrae V1, V2, V3 for receiving the shaft of screw 185. Drilling occurs across facet joint FJ.

Screw 185 is inserted and translated into passageway 184 relative to inner surface 182 to adjacent end 180 in a distal direction via driver 183 described above. Screw 185 rests at portion 190 and is viewed via opening 192 of tube 186, as shown in FIG. 17. In one embodiment, driver 183 can be attached to screw 185 and inserted into sleeve 176 to fix screw 185 within sleeve 176 before screw 185 is disposed adjacent the surgical site. Screw 185 is inserted and fixed within a pilot hole via driver 183. Upon delivery of screw 185 to the surgical site, the surgical instruments, assemblies and non-implant components of system 20 are removed from the surgical site and the incision(s) is closed.

Screw 185 may be employed as a bone screw, pedicle screw, mono-axial screw or multi-axial screw used in spinal surgery. In one embodiment, screw 185 may be coated with an osteoconductive material such as hydroxyapatite and/or osteoinductive agent such as a bone morphogenic protein for enhanced bony fixation. In some embodiments, screw 185 may be coated with therapeutic and/or pharmacological agents for release, including sustained release, to treat, for example, pain, inflammation and degeneration. Screw 185 can be made of radiolucent materials such as polymers. Radiomarkers may be included for identification under x-ray, fluoroscopy, CT, or other imaging techniques. Metallic or ceramic radiomarkers, such as tantalum beads, tantalum pins, titanium pins, titanium endcaps, and platinum wires can be used.

In one embodiment, as shown in FIGS. 18-22, spinal implant system 20, similar to the systems and methods described above, includes a guide 222, similar to guide 122 described above. Guide 222 includes a frame 224 extending between a first end 226 and a second end 228. End 226 is configured for engagement with a guide element and includes an inner surface that defines an opening 227. Opening 227 is configured to facilitate visualization of a surgical site and support of surgical instruments, similar to that described herein.

End 228 is configured for engagement with a guide element, as described herein. End 228 includes a channel 310. In one embodiment, channel 310 may have various cross section configurations, for example, circular, oval, rectangular, polygonal, irregular, tapered, offset, staggered, uniform and non-uniform. Channel 310 includes a proximal portion 312 and a distal portion 314. A lever 318 is connected with an outer surface of channel 310. Lever 318 is rotatable, similar to those mechanisms described above, to lock a guide element with frame 224. In one embodiment, as shown in FIG. 20, a wing bolt 320 is torqued to lock a guide element with frame 224. In one embodiment, one or all of the surfaces of frame 224 may have alternate surface configurations, such as, for example, rough, threaded for connection with surgical instruments, arcuate, undulating, porous, semi-porous, dimpled, polished and/or textured according to the requirements of a particular application.

Guide 222 includes a guide element, such as, for example, a sleeve 276. In one embodiment, sleeve 276 is monolithically formed with end 228. In one embodiment, sleeve 276 is translatable relative to channel 310 for disposal of end 280 adjacent portion 290.

Sleeve 276 has an arcuate configuration and extends between a first end 278 and a second end 280 that defines a distal face including an opening configured for working passage of a surgical instrument and/or an implant. In one embodiment, sleeve 276 may include alternate configurations, such as, for example, linear, offset, staggered and/or an angular orientation.

Sleeve 276 includes an inner surface 282 that defines a passageway 284 having an arcuate configuration. Passageway 284 is configured such that a surgical instrument and/or at least one implant is disposable and/or translatable along passageway 284 relative to inner surface 282 along a selected trajectory. A surgical instrument and/or an implant is introduced into end 278 and passageway 284 is configured as a working channel such that the surgical instrument and/or the implant are translated therethrough and delivered through end 280 to adjacent a surgical site for treating vertebrae. In some embodiments, passageway 284 may have alternate configurations, such as, for example, oval, oblong, triangular, square, polygonal, irregular, uniform, non-uniform, offset, staggered, undulating, variable, V-shaped, and/or tapered depending upon the geometry of the spinal construct to be received within passageway 284 and/or according to a selected trajectory.

System 20 includes a first instrument, such as, for example, a retractor tube 286 connected with frame 224. Tube 286 extends between a first portion 288 and a second portion 290, and defines a first axis c. Portion 288 includes an opening 292. Portion 288 is configured for engagement with opening 227 of frame 224. Portion 288 and opening 227 are configured to facilitate visualization and retraction, similar to that described herein. In one embodiment, portion 288 and opening 227 engage via a friction fitting, snap, bracket, hook, clip, key/keyway clips, threaded engagement and/or male/female engagement. Portion 228 includes an extension, such as, for example, a snake arm 293.

Portion 290 defines a tissue engaging surface 294 having an angled face. Engaging surface 294 is configured to engage with tissue and/or adjacent tissue, such as, for example, a cervical facet. Engaging surface 294 includes a wall surface that defines a cavity, such as, for example, concave opening 300. Opening 300 is configured for disposal of end 280 such that end 280 can be removably supported by tube 286. Tube 286 includes an inner surface that defines a passageway 302.

System 20 including guide 222 is employed, similar to the systems and methods described herein, such that a surgical instrument and/or an implant is introduced into channel 310 and sleeve 276 is configured as a working channel such that the surgical instrument and/or implant are translated therethrough and delivered through end 280 to adjacent a surgical site for treating vertebrae V, which includes vertebra V1, V2, V3, as shown in FIGS. 21-22.

In one embodiment, system 20 including guide 222 is employed for treating vertebrae V1, V2, V3, as shown in FIGS. 21-22, for placing cervical facet screws (not shown) along a selected trajectory to access cervical joints. For example, tube 286 is placed across a facet joint space of vertebra V1. The configuration of guide 222 provides a consistent trajectory for a fixed drill/screw guide.

As shown in FIG. 21, tube 286 is placed over a dilator (not shown) over a facet of vertebra V1. Tube 286 facilitates access through passageway 302 such that a practitioner can open a facet capsule and decorticate the facet. In one embodiment, bone graft may also be introduced adjacent vertebra V1. A curved dilator is inserted within sleeve 276. The assembly of the dilator and sleeve 276 is inserted through channel 310 in a radial fashion with end 280 coming to rest with opening 300, angular such as, for example, perpendicular to the facet and tube 286. The dilator is removed from sleeve 276. A flexible drill shaft 316 having a drill tip 285 is inserted through sleeve 276 to drill across the facet. Tube 286 provides visualization and a bone screw is passed through sleeve 276 with a flexible driver 183 (FIG. 11) across the facet. Upon completion of the procedure, sleeve 276 and tube 286 are removed from the surgical site.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical guide comprising:
a frame connected with a first instrument that defines a first axis and extends to a tissue engaging surface; and
a guide element connected with the frame and including an inner surface that defines a passageway,
wherein the frame is rotatable relative to the first axis to translate the guide element along a trajectory that intersects the first axis such that an end of the guide element is angularly oriented relative to an orientation of the tissue engaging surface and at least one second instrument and/or at least one implant is movable along the passageway and relative to the inner surface to adjacent the tissue engaging surface.

2. A surgical guide as recited in claim 1, wherein the passageway includes an arcuate configuration.

3. A surgical guide as recited in claim 1, wherein the trajectory intersects the first axis adjacent a cervical facet joint.

4. A surgical guide as recited in claim 1, wherein the frame includes a first arm and a second arm spaced apart from the first arm, the arms extending from the guide element to engagement with the first instrument.

5. A surgical guide as recited in claim 1, wherein the frame includes a tensioning member that releasably locks the frame with the first instrument.

6. A surgical guide as recited in claim 1, wherein the frame includes a first arm and a second arm spaced apart from the first arm, the arms extending from the guide element to engagement with the first instrument, the frame further including a transverse tensioning member that connects the arms and has a latch to releasably lock the frame with the first instrument.

7. A surgical guide as recited in claim 1, wherein the frame includes a first arm and a second arm extending from the guide element to engagement with the first instrument, the first arm being configured to pivot relative to the frame.

8. A surgical guide as recited in claim 1, wherein the frame includes at least one mating surface configured to capture and release the first instrument.

9. A surgical guide as recited in claim 1, wherein the frame includes at least one mating surface configured to capture and release the first instrument, the at least one mating surface including a female mating part and the first instrument including a male mating part.

10. A surgical guide as recited in claim 1, wherein the frame includes at least one mating surface configured to capture and release the first instrument, the at least one mating surface including a pair of inwardly oriented ears.

11. A surgical guide as recited in claim 10, wherein the frame includes a thumbwheel connected with an ear and being rotatable to translate the ears into engagement with the first instrument to capture the first instrument.

12. A surgical guide as recited in claim 1, wherein the tissue engaging surface is configured to mate with the end of the guide element.

13. A surgical guide as recited in claim 1, wherein the tissue engaging surface defines a cavity configured for disposal of the end of the guide element.

14. A surgical guide as recited in claim 1, wherein the first instrument is a facet elevator, the at least one second instrument includes a flexible instrument and the at least one implant includes a bone screw.

15. A surgical guide comprising:
a frame extending between a first end and a second end, and having a first arm and a second arm, the first arm being pivotable relative to the second end, the first end being connected with a first instrument that defines a first axis and extends between a first end and a second end configured for engaging tissue, the frame further including a tensioning member that tensions the arms to releasably lock the frame with the first instrument; and
a sleeve connected with the second end of the frame and including an inner surface that defines an arcuate passageway,
wherein the frame is rotatable relative to the first axis to translate the sleeve along a trajectory that intersects the first axis adjacent a spinal facet joint such that an end of the sleeve is angularly oriented relative to an orientation of the second end of the first instrument and at least one second instrument and/or at least one implant is movable along the passageway and relative to the inner surface to adjacent the second end of the first instrument.

16. A surgical guide as recited in claim 15, wherein the first end of the frame includes at least one mating surface configured to capture and release the first end of the first instrument, the at least one mating surface including a female mating part and the first end of the first instrument including a male mating part.

17. A surgical guide as recited in claim 15, wherein the frame includes at least one mating surface configured to capture and release the first end of the first instrument, the at least one mating surface including a pair of inwardly oriented ears.

18. A surgical guide as recited in claim 17, wherein the frame includes a thumbwheel connected with an ear and being rotatable to translate the ears into engagement with the first end of the first instrument to capture the first instrument.

19. A surgical guide as recited in claim 15, wherein the first instrument is a facet elevator, the at least one second instrument includes a flexible instrument and the at least one implant includes a bone screw.

20. A spinal implant system comprising:
a surgical guide including a frame and a guide element connected with the frame and including an inner surface that defines an arcuate passageway;
a facet elevator defining a first axis and extending to a tissue engagement surface;
at least one instrument configured for translation along the passageway; and
at least one bone screw,
wherein the frame is rotatable relative to the first axis to translate the guide element along a trajectory that intersects the first axis such that an end of the guide element is angularly oriented relative to an orientation of the tissue engaging surface and the at least one instrument and/or the at least one bone screw is movable along the passageway and relative to the inner surface to adjacent the tissue engaging surface.
